(19) United States
(12) Patent Application Publication
(10) Pub. No.: US 2022/0253629 A1
(43) Pub. Date: Aug. 11, 2022

(54) HEALTH CARING SYSTEM AND HEALTH CARING METHOD

(71) Applicant: National Tsing Hua University, Hsinchu City (TW)

(72) Inventors: Min Sun, Hsinchu City (TW); Chin-An Cheng, Hsinchu City (TW); Hou-Ning Hu, Hsinchu City (TW)

(73) Assignee: National Tsing Hua University, Hsinchu City (TW)

(21) Appl. No.: 17/321,533

(22) Filed: May 17, 2021

(30) Foreign Application Priority Data
Feb. 9, 2021 (TW) 110104980

(51) Int. Cl. G06K 9/00; G06K 9/62; G06K 9/46; G06T 7/70; G08B 21/04; A61B 5/11; A61B 5/00

(52) U.S. Cl. CPC G06K 9/00335; G06K 9/6215; G06K 9/4661; G06K 9/00362; G06T 7/70; G06T 2207/30196; G08B 21/0415; A61B 5/1116; A61B 5/0033; A61B 5/746; G06K 9/00771

(57) ABSTRACT

A health caring system and a health caring method are provided. The health caring method includes: obtaining image data of a target space and a space division configuration corresponding to the target space, wherein the image data include time information; obtaining a posture of a person according to the image data; determining a space division where the person is located according to the image data and the space division configuration; determining a behavior of the person according to the posture, the space division, and the time information; determining an event has occurred according to the behavior, the space division, and the time information; and outputting an alarm message corresponding to the event.

### CROSS-REFERENCE TO RELATED APPLICATION

This application claims the priority benefit of Taiwan application serial no. 110104980, filed on Feb. 9, 2021. The entirety of the above-mentioned patent application is hereby incorporated by reference herein and made a part of this specification.

### BACKGROUND

### Field of the Disclosure

The disclosure relates to a health caring system and health caring method.

### Description of Related Art

As aging population is growing, an increasing number of elderly people need to receive care services. Currently, there are many health caring systems on the market that can monitor the health condition of users. For most health caring systems, users are required to wear a wearable device to sense the physiological state of the user through a sensor on the wearable device. However, the discomfort caused by the wearable device often makes the user refuse to put on the wearable device. Accordingly, practitioners in the related field are making efforts to find out a method for monitoring the user's state without using a wearable device.

### SUMMARY

The disclosure provides a health caring system and a health caring method that can monitor the status of persons in a target space.

In the disclosure, a health caring system is adaptable for monitoring the state of a person in a target space. The health caring system includes a processor, a storage medium, a transceiver and an image capturing device. The image capturing device captures image data of the target space, where the image data includes time information. The storage medium stores the space division configuration corresponding to the target space. The processor is coupled to the storage medium, the transceiver, and the image capturing device, and is configured to: obtain a posture of a person according to the image data; determine a space division where the person is located according to the image data and the space division configuration; determine a behaviour of the person according to the posture, the space division, and the time information; determine that an event has occurred according to the behaviour, the space division, and the time information; and output an alarm message corresponding to the event through the transceiver.

In an embodiment of the disclosure, the processor creates a virtual identification code corresponding to the person based on the image data, and determines behaviour based on the virtual identification code.

In an embodiment of the disclosure, the processor determines the time period during which the person leaves the space division based on the image data, the space division, and the time information, and determines that the event has occurred in response to the time period being greater than the time threshold.

In an embodiment of the disclosure, the processor determines the time period during which the person performs a behaviour based on the time information, and determines that the event has occurred based on the time period.

In an embodiment of the disclosure, the processor determines that the image data is usable in response to the brightness of the image data being greater than the brightness threshold, and determines that the event has occurred based on the image data in response to the image data being usable.

In an embodiment of the disclosure, the image data includes a first image corresponding to a first time point and a second image corresponding to a second time point, wherein the processor determines that the image data is usable according to the similarity between the first image and the second image, and determines that the event has occurred according to the image data in response to the image data being usable.

In an embodiment of the disclosure, the behaviour includes a first behaviour and a second behaviour, wherein the processor determines the proportion of the first behaviour and the second behaviour in the time period according to the behaviour and the time information, and determines that the event has occurred according to the proportion.

In an embodiment of the disclosure, the processor generates at least one of the following based on the virtual identification code, the behaviour, the space division, and the time information: spatial heatmap, temporal heatmap, trajectory map, action proportion chart, time record of entering space division and time record of leaving space division.

In an embodiment of the disclosure, the storage medium stores historical behaviours corresponding to the person, and the processor determines that the event has occurred based on the historical behaviours and the behaviour.

A health caring method of the disclosure is adaptable for monitoring the status of a person in a target space, including: obtaining the image data of the target space and the space division configuration corresponding to the target space, wherein the image data includes time information; obtaining a posture of a person according to the image data; determining a space division where the person is located according to the image data and the space division configuration; determining a behaviour of the person according to the posture, the space division, and the time information; determining that an event has occurred according to the behaviour, the space division, and the time information; and outputting an alarm message corresponding to the event.

Based on the above, the health caring system of the disclosure can determine the state of the person in the target space by analyzing the image data without using the wearable device.

### BRIEF DESCRIPTION OF THE DRAWINGS

FIG. 1 illustrates a schematic diagram of a health caring system according to an embodiment of the disclosure.

FIG. 2 illustrates a flowchart of a health caring method according to an embodiment of the disclosure.

FIG. 3 illustrates a schematic diagram of image data of a target space according to an embodiment of the disclosure.

FIG. 4 illustrates a schematic diagram of a space division configuration corresponding to a target space according to an embodiment of the disclosure.

FIG. 5 illustrates a schematic diagram of a temporal heatmap according to an embodiment of the disclosure.

FIG. 6 illustrates a schematic diagram of a trajectory map according to an embodiment of the disclosure.

FIG. 7 is a schematic diagram of an action proportion chart according to an embodiment of the disclosure.

FIG. 8 illustrates a flowchart of a health caring method according to another embodiment of the disclosure.

### DETAILED DESCRIPTION OF DISCLOSED EMBODIMENTS

In order to make the content of the present disclosure more comprehensible, the following embodiments are provided as examples based on which the present disclosure can indeed be implemented. In addition, wherever possible, elements/components/steps with the same reference numbers in the drawings and embodiments represent the same or similar components.

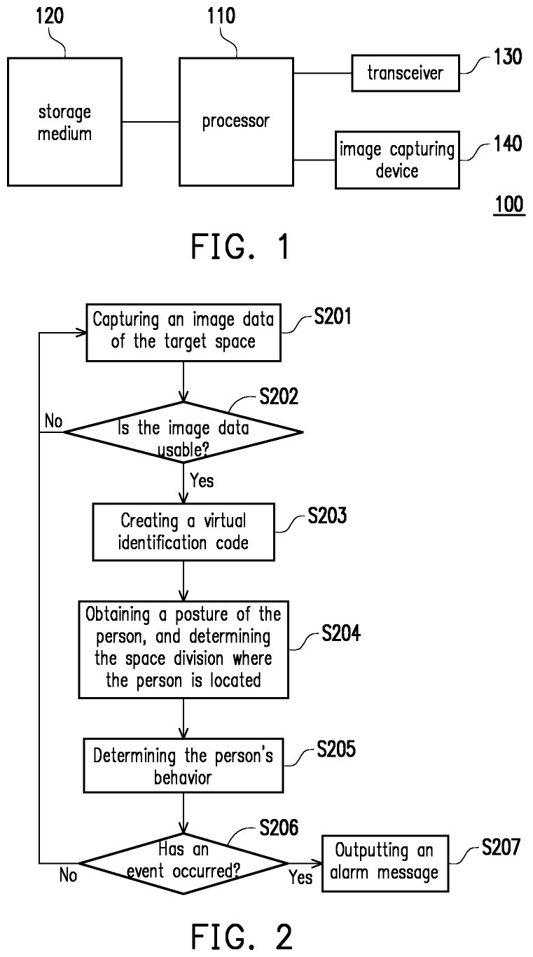

FIG. 1 illustrates a schematic diagram of a health caring system 100 according to an embodiment of the disclosure. The health caring system 100 is adaptable for monitoring the status of persons in the target space. If a specific event has occurred to the monitored person, the health caring system 100 may alert other persons to help the monitored person. In addition, the health caring system 100 can also generate charts related to the health status of the monitored person. The chart can be adopted to assist the user in judging the health status of the monitored person. The health caring system 100 may include a processor 110, a storage medium 120, a transceiver 130, and an image capturing device 140.

The processor 110 is, for example, a central processing unit (CPU), or other programmable general-purpose or specific-purpose micro control unit (MCU), a microprocessor, a digital signal processor (DSP), a programmable controller, an application specific integrated circuit (ASIC), a graphics processing unit (GPU), an image signal processor (ISP), an image processing unit (IPU), an arithmetic logic unit (ALU), a complex programmable logic device (CPLD), a field programmable gate array (FPGA) or other similar components or a combination of the above components. The processor 110 may be coupled to the storage medium 120, the transceiver 130, and the image capturing device 140, and access and execute a plurality of modules and various applications stored in the storage medium 120, thereby realizing the functions of the health caring system.

The storage medium 120 is, for example, any type of fixed or removable random access memory (RAM), a read-only memory (ROM), a flash memory, a hard disk drive (HDD), a solid state drive (SSD) or similar components or a combination of the above components, and configured to store multiple modules or various applications that can be executed by the processor 110 to realize the functions of the health caring system.

The transceiver 130 transmits and receives signals in a wireless or wired manner. The transceiver 130 may also perform operations such as low-noise amplification, impedance matching, frequency mixing, up or down frequency conversion, filtering, amplification, and the like.

The image capturing device 140 can be configured to capture image data of the target space. The target space may be a space where the monitored person often stays. For example, the image capturing device 140 may be installed on the ceiling of the home or office of the person being monitored, so as to capture the image data corresponding to the target space (i.e., home or office). The image data may include images and time information corresponding to the images. In an embodiment, the image capturing device 140 can capture the image data of the target space through a fisheye lens.

FIG. 2 illustrates a flowchart of a health caring method according to an embodiment of the disclosure, wherein the health caring method can be used to monitor the status of a person in the target space, and the health caring method can be implemented through the health caring system 100 as shown in FIG. 1.

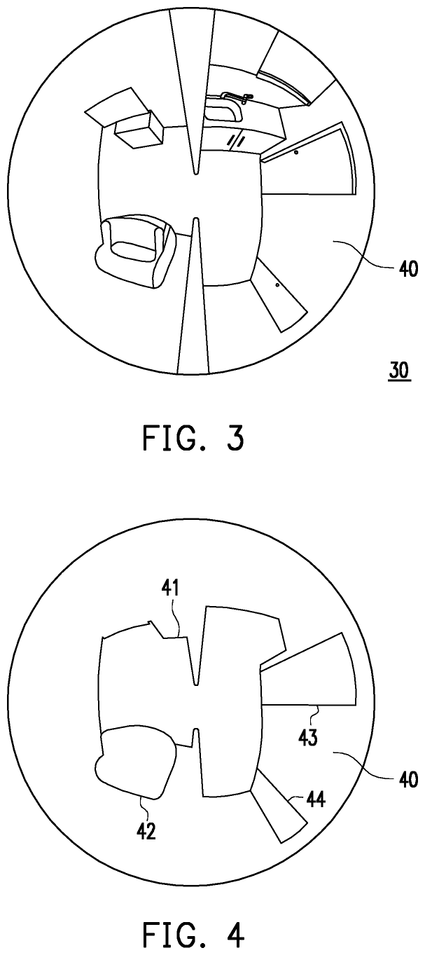

In step S201, the processor 110 of the health caring system 100 may capture image data of the target space through the image capturing device 140, wherein the image data may include the image and time information corresponding to the image. FIG. 3 illustrates a schematic diagram of image data 30 of a target space 40 according to an embodiment of the disclosure. When the image capturing device 140 has a fisheye lens, the image of the target space 40 captured by the image capturing device 140 can be as shown in the image data 30 of FIG. 3. In the embodiment, the target space 40 may include areas such as aisles, sofas, front doors, bathroom doors and so on.

Referring to FIG. 2, in step S202, the processor 110 may determine whether the image data is usable. If the image data is usable, go to step S203. If the image data is not usable, go back to step S201.

In an embodiment, the processor 110 may determine whether the image data is usable according to the brightness of the image data. Specifically, the processor 110 may determine that the image data is usable in response to the brightness of the image data being greater than the brightness threshold, and may determine that the image data is not usable in response to the brightness of the image data being less than or equal to the brightness threshold. In this way, when the image data is not clear due to the low brightness, the processor 110 will not use the image data to determine the status of the person in the target space 40.

In an embodiment, the processor 110 may determine whether the image data is usable according to the similarity between different frames of the image data. Specifically, the image data may include a first image corresponding to a first time point and a second image corresponding to a second time point, wherein the first time point may be different from the second time point. The processor 110 may calculate the similarity between the first image and the second image. The disclosure provides no limitation to the method of calculating the similarity. After obtaining the similarity between the first image and the second image, the processor 110 may determine that the image data is usable in response to the similarity being greater than the similarity threshold, and may determine that the image data is not usable in response to the similarity being less than or equal to the similarity threshold. In this way, if the difference between the different image data is too large, the processor 110 will not use the image data to determine the status of the person in the target space 40.

In step S203, the processor 110 may create a virtual identification code for the person in the target space according to the image data. For example, if a person A and a person B are located in the target space 40, the processor 110 may create a corresponding virtual identification code A for the person A, and may create a corresponding virtual identification code B for the person B.

In step S204, the processor 110 can obtain the posture of the person according to the image data, and can determine the space division where the person is located according to the image data and the space division configuration, wherein the posture of the person is, for example, associated with the articulation point of the person.

Specifically, the storage medium 120 may prestore the space division configuration corresponding to the target space 40. The space division configuration can be adopted to divide the target space 40 into one or more regions. FIG. 4 illustrates a schematic diagram of a space division configuration corresponding to a target space 40 according to an embodiment of the disclosure. In the embodiment, the space division configuration can divide the target space 40 into a space division 41 corresponding to the aisle, a space division 42 corresponding to a sofa, a space division 43 corresponding to a front door, and a space division 44 corresponding to a bathroom door. The processor 110 can determine which space division of the target space 40 that the person is located in according to the image data, so as to determine the location information of the person.

The processor 110 may set the acquired posture or space division and related information to be associated with the virtual identification code. For example, information such as the acquired posture or space division is set to be associated with the virtual identification code A, thereby indicating that the posture or the space division corresponds to the person A.

Referring to FIG. 2, in step S205, the processor 110 may determine the behaviour of the person. Specifically, the processor 110 can determine the behaviour of the monitored person according to the virtual identification code, posture, space division or time information, etc. For example, the processor 110 can determine that the monitored person has been sitting in the space division 42 for several hours based on the virtual identification code, posture, space division, and time information. In this way, the processor 110 can determine that the person's behaviour is “resting on the sofa.”

In step S206, the processor 110 may determine whether an event corresponding to the monitored person has occurred. If an event has occurred, go to step S207. If no event has occurred, return to step S201. Specifically, the processor 110 can determine whether an event corresponding to the monitored person has occurred based on information such as behaviour, space division, or time information.

In an embodiment, the processor 110 may determine the time period during which the person leaves the target space 40 or the space division based on the behaviour, space division, or time information. If the time period is greater than the time threshold, the processor 110 may determine that the event has occurred. For example, the processor 110 may determine that the monitored person has left the target space 40 from the space division 44 representing the bathroom door for more than 1 hour based on the behaviour, space division, or time information. As such, it means that the person has entered the bathroom for more than 1 hour. The person entering the bathroom for more than one hour means that the person might pass out in the bathroom. Therefore, the processor 110 can determine that an event that “person might pass out in the bathroom” has occurred.

In an embodiment, the processor 110 may determine the time period during which a person performs a specific behaviour based on the time information, and determine that the event has occurred based on the time period. For example, the processor 110 may determine, based on the time information, that the time for the person to perform the “lying” behaviour in the space division 41 representing the aisle exceeds 5 minutes. In this way, it means that the person might fall down on the aisle and cannot get up on his own. Therefore, the processor 110 can determine that an event “person fall” has occurred.

In an embodiment, if the person performs multiple behaviours including the first behaviour and the second behaviour, the processor 110 may determine the proportion of the first behaviour and the second behaviour in a specific time period based on the multiple behaviours and time information, and determine that the event has occurred according to the proportion. For example, if the person has performed various behaviours such as “walking” and “lying”, the processor 110 may determine that the person often lies down and lacks exercise in response to the high proportion of the “lying” behaviour and the “walking” behaviour. Based on this, the processor 110 can determine that an event that “person's activity status is different from normal status” has occurred.

In an embodiment, the storage medium 120 may prestore historical behaviours corresponding to the monitored person. The processor 110 can determine that the event has occurred according to the historical behaviours and the current behaviour. For example, the processor 110 can determine that the person's historical daily lying time is about 10 hours based on the person's historical behaviour, and can determine that the person's daily lying time is about 12 hours based on the person's current behaviour. Accordingly, the processor 110 can determine that the person's lying time has increased. Therefore, the processor 101 can determine that an event of “decrease of person's activity” has occurred.

In step S207, the processor 110 may output an alarm message corresponding to the event through the transceiver 130. For example, when the processor 110 determines that the monitored person in the target space 40 has fallen down, the processor 110 may send an alarm message to the family or caregiver of the person through the transceiver 130 to notify the family or caregiver to help the monitored person as soon as possible.

In an embodiment, the processor 110 may generate various charts based on virtual identification codes, behaviours, spatial divisions, or time information, wherein the various charts may include, but are not limited to, a spatial heatmap, a temporal heatmap, a trajectory map, an action proportion chart, time record of entering space division and time record of leaving space division. The processor 110 may output the generated chart through the transceiver 130. For example, the processor 110 may transmit the generated chart to the user's terminal device through the transceiver 130. The user can view the chart through the display of the terminal device.

The spatial heatmap can be used to determine the frequency of monitored persons in different locations. For example, the user of the health caring system 100 can determine that the monitored person frequently appears in the space division 42 within a specific time period according to the spatial heatmap, thereby determining that the person often rests on the sofa.

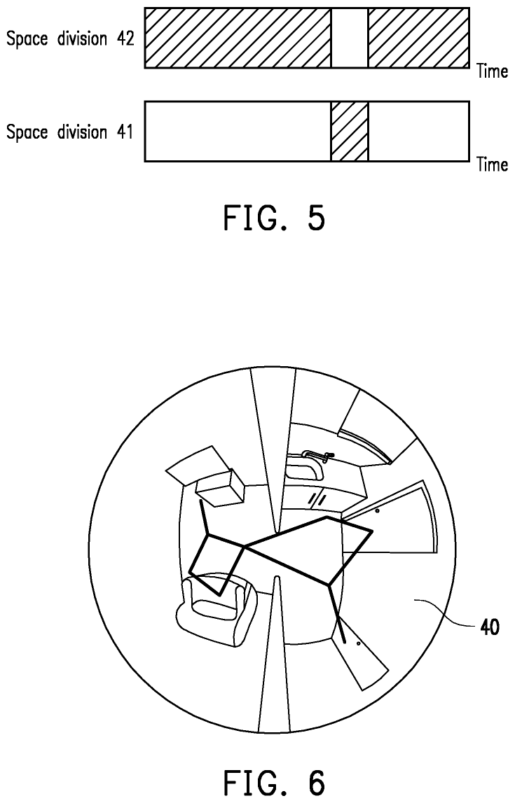

The temporal heatmap can be used to determine the time during which the monitored person is at the location. FIG. 5 illustrates a schematic diagram of a temporal heatmap according to an embodiment of the disclosure. For example, the user of the health caring system 100 can determine that the time during which the monitored person staying in the space division 42 is much longer than the time during which the monitored person staying in the space division 41 according to the temporal heatmap shown in FIG. 5.

The trajectory map can be used to determine the movement trajectory of the monitored person in the target space 40. FIG. 6 illustrates a schematic diagram of a trajectory map according to an embodiment of the disclosure. For example, the user of the health caring system 100 can determine the movement trajectory of the monitored person in the target space 40 according to the trajectory map shown in FIG. 6.

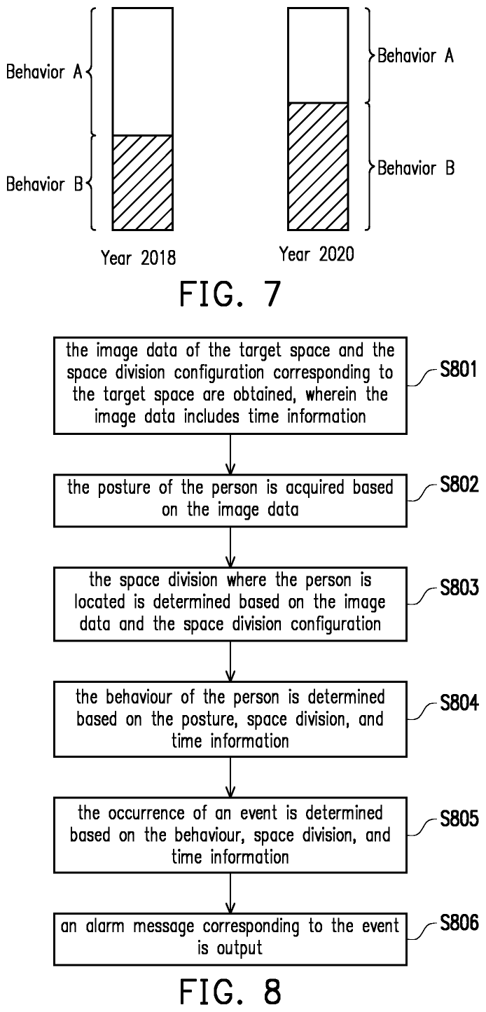

The action proportion chart can be used to determine the proportion of different behaviours performed by the monitored person. FIG. 7 is a schematic diagram of an action proportion chart according to an embodiment of the disclosure. For example, the user of the health caring system 100 can judge from the action proportion chart shown in FIG. 7 that the proportion of the behaviour A performed by the monitored person decreases along with time, and the proportion of the behaviour B performed by the monitored person increases along with time.

The time record of entering the space division and the time record of leaving the space division can be used to determine the time at which the monitored person enters or leaves the space division. For example, the user of the health caring system 100 can determine that the monitored person leaves the space division 44 at 20:00 and returns to the space division 44 at 20:10 according to the time record of entering the space division and the time record of leaving the space division.

FIG. 8 illustrates a flowchart of a health caring method according to another embodiment of the disclosure, wherein the health caring method is adaptable for monitoring the status of persons in the target space, and the health caring method may be implemented through the health caring system 100 shown in FIG. 1. In step S801, the image data of the target space and the space division configuration corresponding to the target space are obtained, wherein the image data includes time information. In step S802, the posture of the person is acquired based on the image data. In step S803, the space division where the person is located is determined based on the image data and the space division configuration. In step S804, the behaviour of the person is determined based on the posture, space division, and time information. In step S805, the occurrence of an event is determined based on the behaviour, space division, and time information. In step S806, an alarm message corresponding to the event is output.

In summary, the health caring system of the disclosure can determine the status of the person in the target space by analyzing the image data obtained by the image capturing device, and the monitored person may not put on a wearable device. The health caring system can determine the posture, position and behaviour of the person in the target space through the image data, and determine whether a specific event has occurred based on the above determining result and time information. If a specific event has occurred, the health caring system can output an alarm message to notify other persons to help the monitored person. The health caring system can also generate corresponding charts for the monitored person. The user can use the chart to determine whether the status of the monitored person is abnormal.

What is claimed is:

1. A health caring system adaptable for monitoring a status of a person in a target space, comprising:
an image capturing device that captures an image data of the target space, wherein the image data comprises time information;
a transceiver;
a storage medium that stores a space division configuration corresponding to the target space; and
a processor that is coupled to the storage medium, the transceiver, and the image capturing device, and is configured to:
obtain a posture of the person according to the image data;
determine a space division where the person is located according to the image data and the space division configuration;
determine a behaviour of the person according to the posture, the space division, and the time information;
determine that an event has occurred according to the behaviour, the space division, and the time information; and
output an alarm message corresponding to the event through the transceiver.

2. The health caring system according to claim 1, wherein the processor creates a virtual identification code corresponding to the person based on the image data, and determines the behaviour based on the virtual identification code.

3. The health caring system according to claim 1, wherein the processor determines a time period during which the person leaves the space division based on the image data, the space division, and the time information, and determines that the event has occurred in response to the time period being greater than a time threshold.

4. The health caring system according to claim 1, wherein the processor determines the time period during which the person performs the behaviour based on the time information, and determines that the event has occurred based on the time period.

5. The health caring system according to claim 1, wherein the processor determines that the image data is usable in response to a brightness of the image data being greater than a brightness threshold, and determines that the event has occurred based on the image data in response to the image data being usable.

6. The health caring system according to claim 1, wherein the image data comprises a first image corresponding to a first time point and a second image corresponding to a second time point, wherein the processor determines that the image data is usable according to a similarity between the first image and the second image, and determines that the event has occurred according to the image data in response to the image data being usable.

7. The health caring system according to claim 1, wherein the behaviour comprises a first behaviour and a second behaviour, wherein the processor determines a proportion of the first behaviour and the second behaviour in a time period according to the behaviour and the time information, and determines that the event has occurred according to the proportion.

8. The health caring system according to claim 2, wherein the processor generates at least one of the following based on the virtual identification code, the behaviour, the space division, and the time information: a spatial heatmap, a temporal heatmap, a trajectory map, an action proportion chart, a time record of entering the space division and a time record of leaving the space division.

9. The health caring system according to claim 1, wherein the storage medium stores a historical behaviour corresponding to the person, and the processor determines that the event has occurred based on the historical behaviour and the behaviour.

10. A health caring method, adaptable for monitoring a status of a person in a target space, comprising:
obtaining an image data of the target space and a space division configuration corresponding to the target space, wherein the image data comprises time information;
obtaining a posture of the person according to the image data;
determining a space division where the person is located according to the image data and the space division configuration;
determining a behaviour of the person according to the posture, the space division, and the time information;
determining that an event has occurred according to the behaviour, the space division, and the time information; and
outputting an alarm message corresponding to the event.
